(19) United States
(12) Patent Application Publication
(10) Pub. No.: US 2008/0243170 A1
(43) Pub. Date: Oct. 2, 2008

(54) EMBOLIC CAPTURING DEVICES AND METHODS

(75) Inventors: Mark L. Jenson, Greenfield, MN (US); William J. Drasler, Minnetonka, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/694,199

(22) Filed: Mar. 30, 2007

(51) Int. Cl. A61M 29/00

(52) U.S. Cl. 606/200

(57) ABSTRACT

Disclosed is an emboli capturing device having a first support hoop at a distal portion of a first elongate member, and a support member that is coupled to the first support hoop. The support member can comprise a second elongate member. In some cases, the second elongate member can be coupled to the first support hoop. In other cases, the support member can comprise a second elongate member and a support strut. The support strut can be coupled to both the first support hoop and the second elongate member. Movement of the support member relative to the first elongate member can translate the first support hoop between collapsed and expanded configurations. Once in a desired configuration, the support members can also provide support to maintain the first support hoop in the desired configuration. Also disclosed is a combination emboli capturing and perfusion device. The combination device can have a perfusion member in combination with an emboli capturing device. The perfusion member can facilitate the introduction of fluids around a lesion in a vessel of a patient, for example if the lesion is creating a partial or total occlusion of the vessel.

### TECHNICAL FIELD

The technical field pertains generally to the design and use of embolic capturing devices.

### BACKGROUND

Emboli such as thrombi or other foreign matter can cause a variety of medical issues, including partial or total occlusion of body lumens. Such an occlusion in vasculature can lead to blockage of blood flow, or ischemia, which can result in conditions such as a stroke or an infarction. As a result, it is often desirable to remove foreign matter from a body lumen. Emboli can appear in a patient's body in a variety of forms. For example, emboli can be entrained in the blood or other fluids of a patient, or emboli can be adhered to the walls of body lumens, among other possibilities. A number of different embolic capturing devices and methods are known, each having certain advantages and disadvantages. However, there is an ongoing need to provide alternative structures, assemblies and methods for capturing emboli.

### SUMMARY OF SOME EMBODIMENTS

An example embodiment can be found in an emboli capturing device having a first elongate member with a distal portion. The emboli capturing device can also have a first support hoop coupled to the first elongate member distal portion and the first support hoop can have a collapsed configuration and an expanded configuration. A capture membrane can be disposed on the support hoop, forming a capture basket. The emboli capturing device can also have a first support member comprising a second elongate member with a distal end and the first and second elongate members can be longitudinally translatable with respect to one another. The first elongate member can be coupled to the first support hoop at a first location on the first support hoop and the first support member can be coupled to the first support hoop at a second, different location on the first support hoop.

The first support member can also comprise a support strut, for example a second support hoop. In some embodiments the second elongate member can be coupled to the first support hoop, and in other embodiments the support strut (e.g., the second support hoop) can form a connection between the second elongate member and the first support hoop.

In another example embodiment, a combination emboli capturing and perfusion device can have both an emboli capturing device and a perfusion member. The emboli capturing device can be, for example, any of the emboli capturing devices discussed herein. The perfusion member can have collapsed and expanded configurations. In the collapsed configuration, the perfusion member can be disposed around one or more of the elongate members of the emboli capturing device. In the expanded configuration the perfusion member can be disposed eccentrically along the one or more of the elongate members, and can define a perfusion lumen. The perfusion lumen can allow fluids to be perfused around an occlusion in a patient's vasculature.

### BRIEF DESCRIPTION OF THE FIGURES

FIG. 1A is a perspective view of an example embolic capturing device support structure in a collapsed configuration;

FIG. 1B is a perspective view of the embolic capturing device support structure of FIG. 1A in an expanded configuration;

FIG. 1C is a cross-sectional view of the embolic capturing device of FIGS. 1A and 1B in an expanded configuration;

FIG. 2 is a perspective view of the embolic capturing device support structure of FIGS. 1A-1C with a filter membrane disposed on the support structure;

FIG. 3A shows the embolic capturing device of FIGS. 1A-1C and 2 being advanced through a body vessel;

FIG. 3B shows the embolic protection device of FIGS. 1A-1C and 2 deployed adjacent embolic material in a body vessel;

FIG. 3C shows the embolic protection device of FIGS. 1A-1C and 2 being used to capture the embolic material in the body vessel;

FIGS. 4-7 are perspective views of alternative embodiments of the embolic capturing device;

FIG. 8 is a perspective view of a combination embolic capturing and perfusion device with both the embolic capturing structure and the perfusion structure in a collapsed configuration;

FIG. 9 is a perspective view of the combination embolic capturing and perfusion device with both the embolic capturing structure and the perfusion structure in a collapsed configuration;

FIG. 10 is a perspective view of an example of a hub that can be disposed at the proximal end of an emboli capturing device; and

FIG. 11 is a perspective view of an example of a filter with a sheath.

### DETAILED DESCRIPTION OF SOME EMBODIMENTS

For the following defined terms, these definitions shall be applied, unless a different definition is given in the claims or elsewhere in this specification.

The term “polymer” will be understood to include polymers, copolymers (e.g., polymers formed using two or more different monomers), oligomers and combinations thereof, as well as polymers, oligomers, or copolymers that can be formed in a miscible blend by, for example, coextrusion or reaction, including transesterification. Both block and random copolymers are included, unless indicated otherwise.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a” “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative embodiments of the claimed invention.

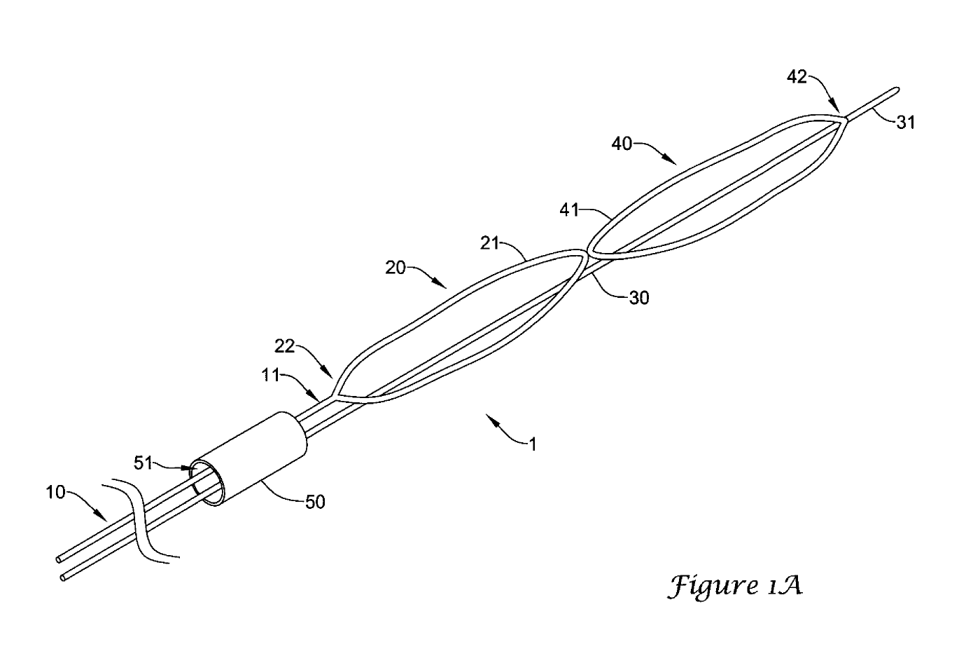

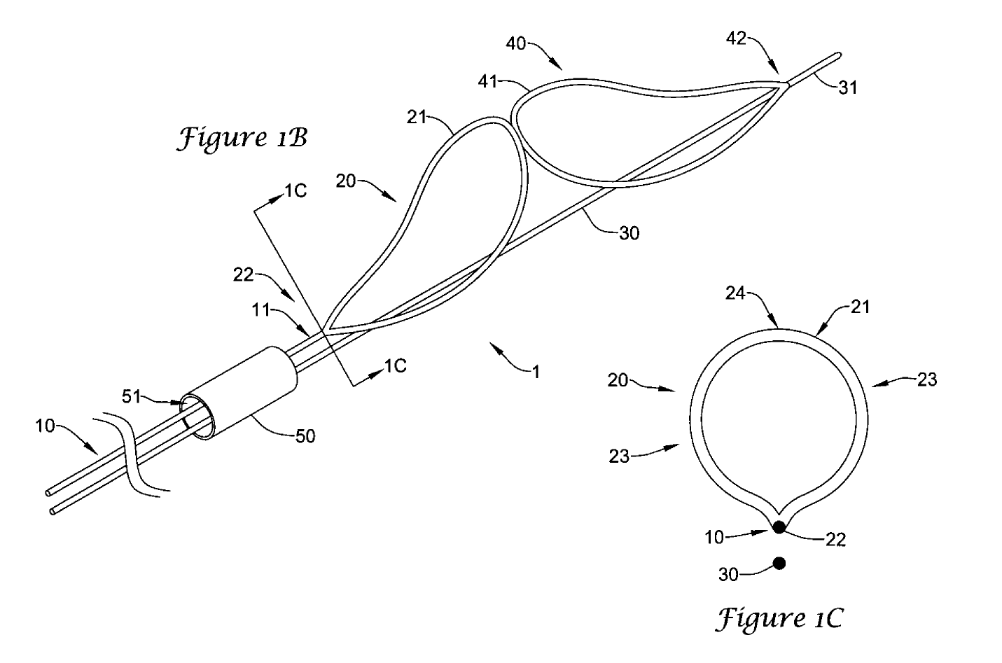

Turning to FIG. 1A, a perspective view of a frame of an example embolic capturing device 1 is shown in a collapsed configuration. The capturing device 1 can have a first elongate member 10 with a distal portion including a distal end 11. Coupled to the first elongate member 10 distal portion (e.g., proximate the first elongate member distal end 11) can be a first support hoop 20. As shown, the first support hoop 20 can also be coupled at the distal end 11.

The first support hoop 20 can include a wire 21 that can have two ends. The wire 21 can be formed into the shape of the first support hoop 20, with the two ends coming together to form a base 22 of the first support hoop 20 (the base 22, sides 23 and the top 24 of the first support hoop 20 are shown and further described with respect to FIG. 1C below). The base 22 of the first support hoop 20 can also be defined as the point at which the first support hoop 20 is coupled to the first elongate member 10. Further, in some embodiments (not shown) the first support hoop 20 can comprise multiple elongate elements. The multiple elongate elements can be joined to form the first support hoop 20. For example, the first support hoop 20 can comprise two elongate elements (e.g., wires). Each one of the elongate elements can form one side 23 of the first support hoop 20. In one embodiment, the two elongate elements can be joined at two points; they can be joined at the base 22 and the top 24 of the first support hoop 20.

Further, it is also contemplated that the first support hoop 20 can be integral with the first elongate member 10. For example, the first elongate member 10 can comprise a first wire that can extend along at least a portion of the length of the first elongate member 10. Further, this first wire can extend into and form at least a portion of the first support hoop 20. As such, a distal end of the first wire can form all or a portion of the first support hoop. For example, the distal end of this first wire can be looped back upon itself and attached to itself, forming the first support hoop 20. The point of attachment of the distal end of the first wire to itself can form the base 22 of the first support hoop 20.

In other embodiments, the first wire can form one side 23 of the first support hoop 20 and an additional wire can form the other side 23 of the first support hoop 20. A distal portion of this additional wire can be attached at a first attachment point to the first wire, forming the top 24 of the first support hoop 20. The first attachment point can be located proximate, or at, the distal end of the first wire. A proximal portion of this additional wire can be attached to the first wire proximal of the first attachment point, forming the base 22 of the first support hoop 20. In such embodiments where the first support hoop 20 is integral with the first elongate member 10, the first elongate member distal end 11 can be defined as a point at, proximate, immediately proximal, or proximal, the base 22 of the first support hoop 20.

In the collapsed configuration shown in FIG. 1A, the first support hoop 20 can generally be disposed approximately parallel to a longitudinal axis along the first elongate member 10. The disposition (e.g., parallel, perpendicular, etc) of the first support hoop 20 (and in some cases the support strut 40, as discussed below), can refer to the disposition of a plane that is formed by the support hoop. As such, when the first support hoop 20 is described as being substantially parallel to an axis, it can refer to a plane that is formed by the open area inside the first support hoop 20 being substantially parallel to the axis. In other cases, it can refer to individual elements of a support hoop being disposed substantially parallel to an axis. Different configurations of the first and/or the second support hoop (20, 40) are discussed below.

The emboli capturing device of FIGS. 1A-1C can also have a support member 30. The support member 30 can include a second elongate member 31 that has a distal portion including a distal end 32. In the embodiments of FIGS. 1A-1C, the support member 30 also comprises a support strut 40, in this case shown as a second support hoop 40. The support strut 40 can extend from the second elongate member 31 to the first support hoop 20. In cases where the support strut 40 is a second support hoop, the second support hoop 40 can be similar in any respect to the first support hoop 20. One portion of the support strut 40 (e.g., the base 42 of the second support hoop 40) can be attached to the second elongate member distal portion (e.g., proximate the second elongate member distal end 32). Another portion of the support strut 40 can be attached to the first support hoop 20. In a collapsed configuration (e.g., see FIG. 1A), the support strut 40 can be disposed substantially parallel to a longitudinal axis of the first elongate member 10, the second elongate member 31, or both. For example, the plane formed by the opening defined by the second support hoop 40 shown in FIGS. 1A and 1B can be substantially parallel to the first and/or the second elongate members (10, 31).

In other examples, one or more elements of the support strut 40 can be substantially parallel with the first and/or the second elongate members (10, 31). The construction of the support member 40 (or multiple support members) is further discussed below.

In some cases, as shown in FIGS. 1A and 1B, the first and second elongate members (10, 31) can extend proximally parallel to one another, adjacent one another, or, as shown in FIGS. 1A and 1B, both parallel and adjacent one another, for at least part of their lengths. These elongate members (10, 31) can be longitudinally translatable with respect to one another. In some cases, an alignment member 50 can be used to maintain the elongate members (10, 31) generally adjacent or parallel, or both adjacent and parallel, to one another. The alignment member could be, for example, a ring, a collar, or a tubular member, or any other structure that can facilitate the alignment of the elongate members. The alignment member 50 can have a lumen 51 in which both elongate members (10, 31) can be disposed. In order to maintain the longitudinal position of the alignment member 50, it can be coupled to and/or longitudinally fixed along the first elongate member 10 or to the second elongate member 31. For example, it could be coupled to the first or second elongate member proximate the first elongate member distal end 11.

Further, the alignment member 50 can be an elongate member that can extend proximally, for example proximally from a point proximate the first elongate member distal end 11. In one embodiment, the alignment member 50 could extend only a short distance proximally. Alternatively, the alignment member 50 could take the form of a tubular member such as a catheter-like structure. In some cases, the tubular member could extend proximally to, or proximate, the proximal end of the first elongate member 10, the proximal end of the second elongate member 31, or the proximal end of both elongate members. Also, the alignment member 50 can have separate lumens for each elongate member (10, 31), or the alignment member 50 could be formed around either the first or second elongate member (10 or 31) and define a lumen in which the other s elongate member (10 or 31) can be disposed.

In addition, the movement of the first and second elongate members (10, 31) can facilitate movement of the first support hoop 20 between a collapsed configuration and an expanded configuration. FIG. 1A shows the first support hoop 20 and the support strut 40 in a collapsed configuration and FIG. 1B shows these structures in an expanded configuration. In this embodiment, in the collapsed configuration, the first support hoop 20 and the support strut 40 can be substantially parallel to the longitudinal axis of the first elongate member 10 or the longitudinal axis of the second elongate member 31, or both. When moved into an expanded configuration, the first support hoop 20 and the support strut 40 can move from a substantially parallel configuration to being disposed at an angle away from the longitudinal axis of the first elongate member 10 or the second elongate member 40, or both. In some cases, the first support hoop 20 and the support strut 40 can have a collapsed configuration in which they are disposed at a first angle from the longitudinal axis of the first elongate member 10 or the second elongate member 31, or both. In such a case, the first support hoop 20 and the support strut 40 can also have an expanded configuration in which they are disposed at a second, larger angle from the longitudinal axis of the first elongate member 10 or the second elongate member 31, or both.

In the example of FIGS. 1A-1C, the support strut 40 can facilitate the translation or actuation of the first support hoop 20 between collapsed and expanded configurations. The support strut 40 can also provide support for maintaining the first support hoop 20 in a collapsed configuration, in an expanded configuration, or both. For example, in FIGS. 1A-1C, as the second elongate member 31 is translated proximally with respect to the first elongate member 10 from a first distal position to a second proximal position, the support strut 40 can transmit force from the second elongate member 31 to the first support hoop 20, which can cause the first support hoop 20 to move from a collapsed configuration toward an expanded configuration. In the embodiment shown in FIGS. 1A-1C, the support strut 40 provides a pushing force that can move the first support hoop 20 from a collapsed configuration to an expanded configuration. In addition, if the second elongate member 31 is maintained in the second position, the second elongate member 31 can provide support for the first support hoop 20 to be held in an expanded configuration.

Also, the second elongate member 31 can be used to move the first support hoop 20 from an expanded configuration to a collapsed configuration. The second elongate member 31 can be moved distally with respect to the first elongate member 10 from a first proximal position to a second distal position, which can place a force on the first support hoop 20 (the force can be transmitted through the support strut 40) that tends to extend or stretch out the first support hoop 20 (and the support strut 40). This can collapse the first support hoop 20 (and the support strut 40) toward the longitudinal axis of the first and/or second elongate members (10, 31). In addition, the extension or stretching force on the first support hoop 20 and the support strut 40 can cause the sides 23 of the first support hoop (and the sides of the second support hoop 40, when present) to collapse toward one another, in some cases providing for a lower overall profile of the emboli capturing device. In some cases, the emboli capturing device can also be held in a collapsed configuration by holding the second elongate member 31 in a distally extended position relative to the first elongate member 10.

Additionally, the first support hoop 20 or the support strut 40, or both, can be predisposed to assume an expanded configuration or a collapsed configuration. In such cases, movement of the second elongate member 31 (and the resulting forces that can be transmitted to the first support hoop 20, the support strut 40, or both) can assist in overcoming such predispositions. For example, if the first support hoop 20, the support strut 40, or both, were predisposed to assume an expanded configuration, movement of the second elongate member 31 distally as described herein could be used to move (and in some cases hold) the first support hoop 20, the support strut 40, or both, toward a collapsed configuration. The opposite is also contemplated; if the first support hoop 20, the support strut 40, or both, were predisposed to assume a collapsed configuration, movement of the second elongate member 31 proximally as described herein could be used to move (and in some cases hold) the first support hoop 20, the support strut 40, or both, toward an expanded configuration.

Turning to FIG. 1C, a cross-sectional view of the first support hoop 20 is shown. This figure shows the first support hoop base 22 along with the sides 23 of the first support hoop 20, and a top 24 of the first support hoop 20. In order to facilitate the transfer of force from the support member 30 to the first support hoop 20, the support member 30 can be coupled to at or near the top 24 of the first support hoop 20. As shown in FIGS. 1A and 1B, the support member 30, for example the support strut 40, can be coupled to the top 24 of first support hoop 20. In other embodiments, the support member 30 (e.g., a portion of the support member 30 such as the support strut 40) can be coupled to one or both sides 23 of the first support hoop 20. In cases where the support strut 40 is a second support hoop, the second support hoop can be similar in any respect to the first support hoop 20.

In some cases, the support member 30 can comprise two or more (e.g., 2, 3 or 4) support struts extending from the second elongate member 31 to the first support hoop 20. In some embodiments, the multiple support struts can each be coupled to a first support hoop side 23. In some cases, the support strut or struts can comprise elongate members, for example elongate wires, that can extend from the second elongate member 31 to the first support hoop 20.

Further, the support hoops or struts can incorporate designs that allow the hoops or struts to collapse in a predictable manner. For example, the hoops or struts could have articulation regions that can cause the hoop or strut to collapse in a predictable manner when the hoop or strut is bent or otherwise placed under stress. The articulation regions could be areas of reduced dimension (e.g., reduced diameter, reduced cross-sectional area, or other dimension). One or more articulation regions could be, for example, at the top 24 of the first support hoop 20 or on the sides 23 of the first support hoop 20, or both at the top 24 and on one or both sides 23. One or more articulation regions could also be located at similar locations on the support strut 40, for example the second support hoop 40. Further examples of such articulation regions are given in U.S. Pat. No. 6,589,263 to Hopkins et al., which is hereby incorporated by reference in its entirety.

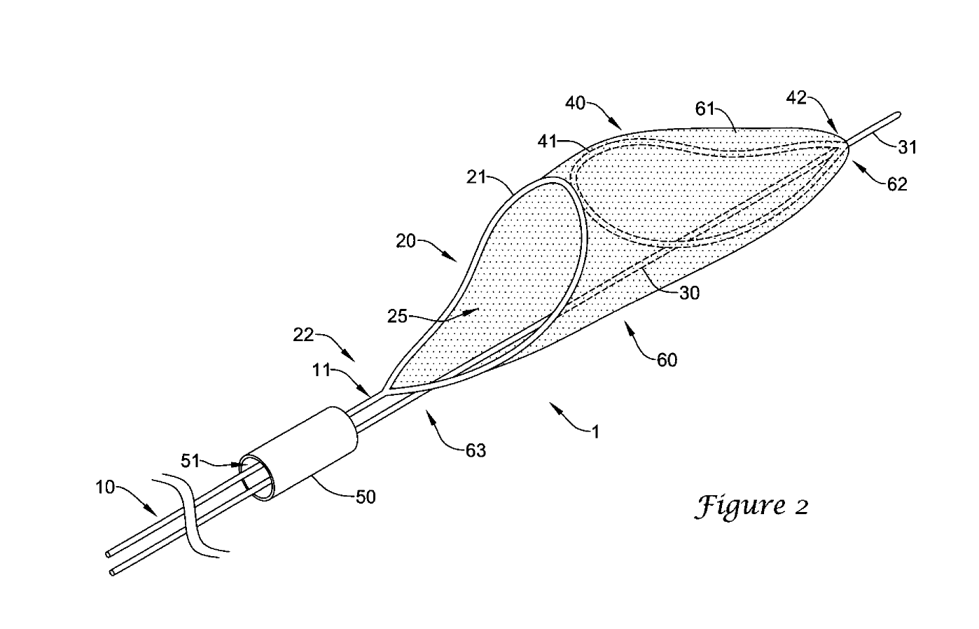

Turning to FIG. 2, an embolic capturing device support frame is shown with a capturing membrane 61 disposed thereon. In this example, the support frame that is shown is the same as the support frame shown in FIGS. 1A-1C. The capturing membrane 61 defines a capturing basket 60. When the embolic capturing device support frame is in its expanded configuration, as shown in FIG. 2, the capturing basket 60 can be configured to receive and capture emboli. In some cases, the capturing membrane 61 can be substantially impervious to the flow of bodily fluids such as blood. In other cases, the capturing membrane 61 can be porous. For example, the capturing membrane 61 can be made at least partially of a material through which fluids can flow. In other cases, the capturing membrane 61 can have pores formed in it, for example such holes could be formed with laser. Porous embodiments of the capturing membrane 61 can have pores that are sized to allow blood flow through the pores while being small enough to prevent emboli from flowing through the pores.

The capturing membrane 61 can have a distal end 62 and a proximal end 63. The capturing membrane proximal end 63 can be attached to the first support hoop 20, and the first support hoop 20 can form a mouth 25 of the capturing basket 60. The first support hoop 20 can be disposed inside the capturing membrane 61, outside the capturing membrane 61, or on the proximal extremity of the capturing membrane 61. Further, the capturing membrane 61 can be attached to the first support hoop 20, for example at one or more discrete points, or along a length of, the first support hoop 20.

As shown in FIG. 2, the capturing membrane 61 can extend distally from the first support hoop 20, for example in a tapering fashion. The distal end of the capturing membrane 61 can be attached to the second elongate member 31, in some cases proximate, and in other cases at, the base 42 of the support strut 40. In other embodiments, the capturing membrane 61 can be attached to the second elongate member 31 proximal or distal of the base 42 of the support strut 40. The support strut 40 (or struts) can be disposed inside the capturing basket 60 (as shown in FIG. 2), or outside of the capturing basket 60. In some cases, the support strut 40 (or struts) can be attached to the capturing basket 60, for example at one or more discrete points, or along a length of, the support strut 40 (or struts).

Further, in some cases the second elongate member 31 can be disposed inside the capturing basket 60, for example for the entire length of the capturing basket 60 (which can be from the proximal end 63 to the distal end 62 of the capturing membrane 61). Alternatively, the second elongate member 31 can be disposed outside of the capturing basket 60, for example along the entire length of the capturing basket 60. In other embodiments, the second elongate member 31 can be disposed inside of the capturing basket 60 for a portion of the length of the capturing basket 60 and outside of the capturing membrane 61 for a portion of the length of the basket 60. In any case, the capturing membrane 61 can be attached to the second elongate member 31 along all or a portion of the length of the capturing membrane 61.

The emboli capturing devices described in this application can be used in a variety of ways. For example, if the capturing membrane 61 is at least partially porous with respect to bodily fluids such as blood, the capturing device can be used in procedures where fluid filtration is involved. In such a procedure, the emboli capturing device can be placed in a patient's vasculature at a desired location, and the emboli capturing device can be deployed from a collapsed configuration to an expanded configuration, as described herein. In some cases, in an expanded configuration, the capturing device can fill most, or all, of the cross-section of a body vessel (e.g., a blood vessel), essentially filtering most, or all, of any fluids (e.g., blood) flowing through the body vessel. Embolic material entrained in the fluids can be filtered out as the fluids are passed through the capturing device. When the filtration procedure is completed, the capturing device can be moved from an expanded configuration to a collapsed configuration as described herein, in some cases maintaining all or a portion of the emboli within the capturing device. The capturing device can then be removed from the patient along with the captured emboli.

In some cases, the capturing devices, when deployed in an expanded configuration, can substantially assume the shape and size, or even form a seal against, an inner wall of a body vessel. The capturing devices can maintain this position and can be resistant to collapse, even under the influence of high speed and/or volume fluid flow or other outside forces. For example, in the presence of such forces, the support members discussed herein (e.g., the combination of a second (and sometimes a third or more) elongate member with, in some cases, one or more support struts) can facilitate the maintenance of the capturing device in an expanded configuration.

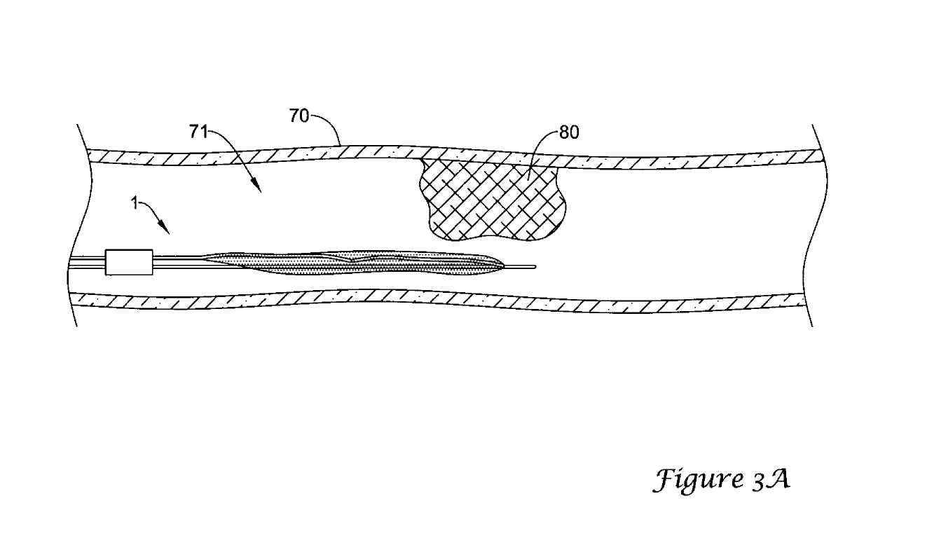

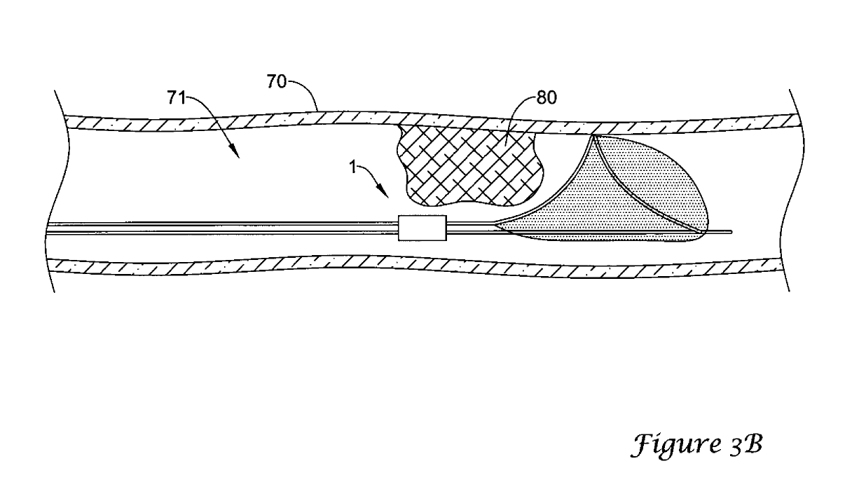

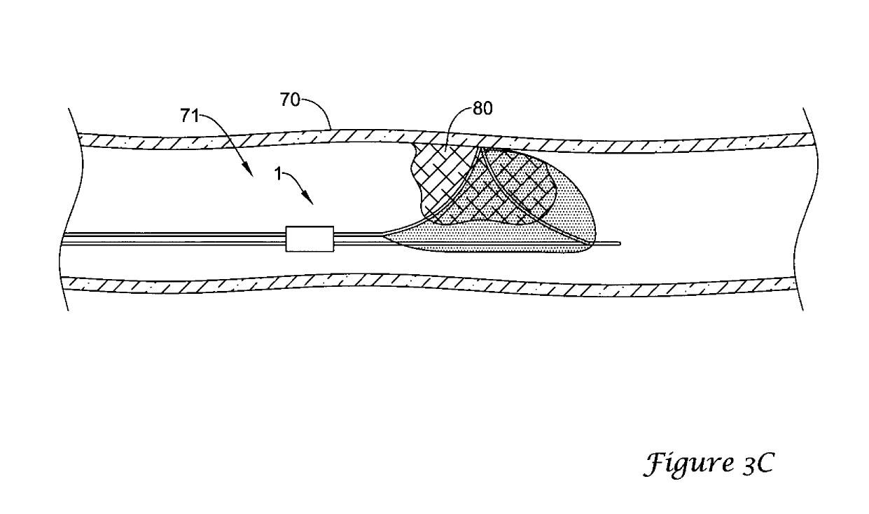

FIGS. 3A-3C show another possible method of use for the capturing devices of this application. In FIG. 3A, any of the emboli capturing devices described herein (this Figure shows as an example the devices described with respect to FIGS. 1A-1C) can be advanced through the vasculature of a patient. As shown, the emboli capturing device 1 can be advanced through the vasculature of a patient in a collapsed configuration. In some cases, the lower profile of the collapsed configuration of the emboli capturing device can facilitate the movement of the emboli capturing device through the vasculature. As shown in FIGS. 3A-3C, a vessel 70 of a patient can define a vessel lumen 71 that is partially or entirely blocked with an embolus 80.

In FIG. 3B, the emboli capturing device 1 is shown advanced to a point distal of a treatment site (e.g., the location of the embolus 80) and deployed from a collapsed configuration to an expanded configuration, for example by any of the methods discussed herein. Any of the emboli capturing devices described herein can be deployed using any appropriate method, for example the methods discussed herein. In some cases, the emboli capturing devices can also be partially or entirely self-expanding, and as such the elongate members can be used to hold the emboli capturing device in a collapsed configuration and, when the elongate members release the emboli capturing device the emboli capturing device can partially or entirely assume an expanded configuration. In such cases, one or more of the elongate members can then assist the emboli capturing device in assuming the expanded configuration and/or help hold the emboli capturing device in an expanded configuration. The emboli capturing devices can also be partially or entirely predisposed to assuming a collapsed configuration, and as such the elongate members can be used to hold the emboli capturing device in an expanded configuration and, when the elongate members release the emboli capturing device the emboli capturing device can partially or entirely assume a collapsed configuration. In such cases, one or more of the elongate members can then assist the emboli capturing device in assuming the collapsed configuration and/or help hold the emboli capturing device in an expanded configuration.

As shown in FIG. 3C, the emboli capturing device 1 in an expanded configuration can be moved in a proximal direction, allowing the embolus 80 to be captured inside the emboli capturing device. In some cases, emboli can resist being captured within the emboli capturing device 1. For instance, if the size of an embolus is close to (or larger than) the size of the mouth of the capturing device, it can be difficult to get the embolus to enter the capturing device. Further, as shown in FIG. 3C, the embolus can be attached to the inner wall of the vessel 70. In order to capture the embolus, the emboli capturing device 1 in some cases must be able to exert sufficient force at the interface between the embolus and the vessel wall so that the embolus 80 will become dislodged, allowing its capture in the emboli capturing device 1. Such applications may tend to collapse the emboli capturing device 1. As an example, when the top 24 of the first support hoop 20 comes into contact with the interface between the vessel wall and the embolus 80 and the first support hoop 20 is urged further proximally in order to separate the vessel wall and the embolus 80, the first support hoop 20 may have a tendency to deflect toward a collapsed configuration. The support members described herein can in some cases help prevent the deflection of the emboli capturing device from an expanded configuration to a collapsed configuration.

Visualization of the emboli capturing device and/or the embolus can also facilitate the removal of emboli. For example, portions of the emboli capturing device can be made radiopaque in order to facilitate visualization of part of all of the emboli capturing device during placement and manipulation of the emboli capturing device during any of the procedures described herein. In some cases, all or a portion of the first support hoop can be made radiopaque. It is also contemplated that portions of the first and/or second elongate members and/or a support strut can also be made radiopaque. These portions of the emboli capturing device can be made radiopaque by fabricating these structures from radiopaque materials. In addition, radiopaque markers (e.g., bands) can be added at any of the above locations.

Further, any of the methods of capturing emboli discussed herein can include the step of introducing contrast media to the location of interest. The contrast media can be introduced before, simultaneously with, or after, the introduction of the emboli capturing device to the point of interest. The contrast media can be used to visualize the emboli and/or the emboli capturing device, facilitating the positioning of the emboli capturing device and the removal of the emboli. In some cases, one or more of the elongate members can define an infusion lumen through which contrast media can be infused to the target site.

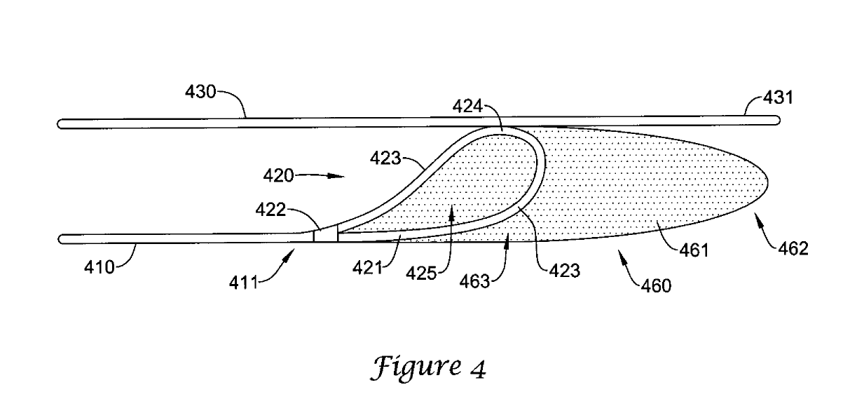

Turning to FIGS. 4-7, some alternative emboli capturing device support frames are shown. These alternatives can have support members that comprise a second elongate member, wherein the second elongate member can be coupled to (attached directly to) the first support hoop.

In FIG. 4, an embodiment of a support frame is shown that comprises a first elongate member 410, a first support hoop 420 forming a mouth 425 of the emboli capturing device, and a support member comprising a second elongate member 430. The first elongate member 410 can have a distal portion with a distal end 411. The first support hoop 420 comprises one or more wires 421, and the first support hoop 420 has a base 422, sides 423 and a top 424. The first elongate member 410 and the first support hoop 420 can be similar in any respect to the first elongate member 10 and the first support hoop 20 described above with respect to FIGS. 1A-1C.

The second elongate member 430 can have a distal portion with a distal end 432. In this example embodiment, the first support hoop 420 is coupled to the second elongate member 430 proximal the second elongate member distal end 432. The first support hoop 420 can also be coupled to the second elongate member 430 proximate, or at, the second elongate member distal end 432. The support hoop 420 can be coupled to the second elongate member 432 at or near the top 424 of the support hoop 420 (as shown) or on one of the sides 423.

The emboli capturing device also has a capture membrane 461 that forms a capture basket 460. The capture membrane 461 has a distal end 462 and a proximal end 463. The capture membrane 461 can be similar in any respect to the capture membrane 61 described with respect to FIG. 2. In the embodiment of FIG. 4, the second elongate member 430 is disposed inside of the capture membrane 461 along the entire length of the capture basket 460. As mentioned with respect to FIG. 2 the second elongate member 430 can also be disposed outside of the capture basket 460 along the length of the capture basket 460, or it could be disposed inside for a portion of the length of the capture basket 460 and outside for a portion.

As shown in FIG. 4, the second elongate member 430 extends distally beyond the first elongate member distal end 411. In some cases, the first elongate member distal end 411 can extend beyond the second elongate member distal end 432 when the emboli capturing device is in a collapsed configuration, in an expanded configuration, or both. In some cases, the first elongate member 410 can extend distally beyond the base 422 of the first support hoop 420, forming a first elongate member extension. The extension could extend distally inside the filter basket 460, outside the filter basket 460, or partially inside and partially outside the filter basket 460. The extension could extend along the entire length of, or only part of the length of, the filter basket 460. Further, the filter basket 460 could be attached to the extension along all, or just a portion of, the extension.

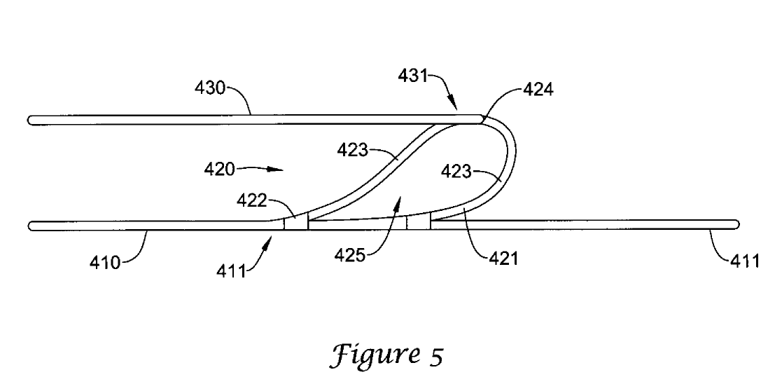

Turning to FIG. 5, a variation of the emboli capturing device support structure shown in FIG. 4 is shown. In this embodiment, the first elongate member distal end 411 extends beyond the second elongate member distal end 432. As shown, the second elongate member distal end 432 can be coupled to the top 424 of the first support hoop 420.

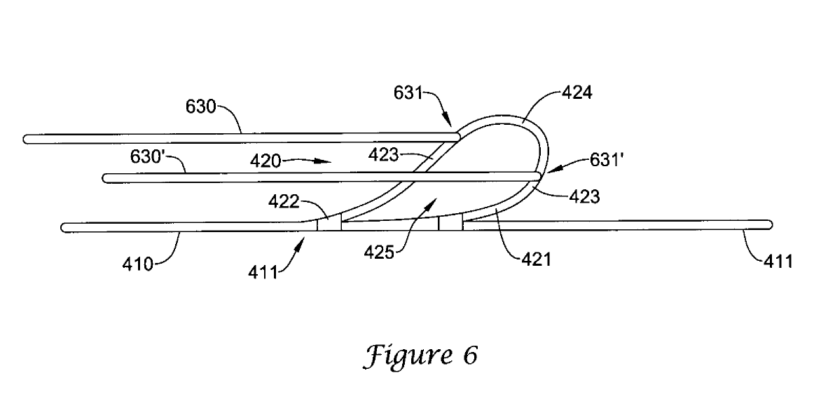

Further, any of the embodiments described herein can include multiple support members, and each support member 30 can comprise an elongate member. As shown in FIG. 6, a first support member 30 comprising a second elongate member 630 and a second support member 30′ comprising a third elongate member 630′ can be included in the emboli capturing device support structure. These elongate members (630, 630′) can be of similar construction and can be incorporated into the emboli capturing device in a manner similar to that described with respect to any of the second elongate members included herein. In the example shown in FIG. 6, the second and third elongate members (630, 630′) can each be coupled to a side 423 of the first support hoop 420. The coupling of the first and second elongate members (630, 630′) along the sides 423 of the first support hoop 420 (rather than the top 424) can facilitate access to the mouth of the emboli capturing device. The second and third elongate members (630, 630′) can have distal ends (632, 632′, respectively). These distal ends (632, 632′) can be coupled to the first support hoop 420 (e.g., the sides 423 of the first support hoop 420). Further, as mentioned herein with respect to second elongate members, the distal ends (632, 632′) can extend distally in a variety of configurations with respect to the first elongate member 410, the first support hoop 420 or a filter basket. In some embodiments, the first support hoop 420 can be coupled to one or both of the elongate members proximal the elongate member distal ends (632, 632′). Also, it is contemplated that the support structure could comprise additional support members, for example 3, 4, 5, or 6 support members. As described with respect to FIGS. 2 and 4, the frame of FIG. 6 can have a capturing basket disposed thereon.

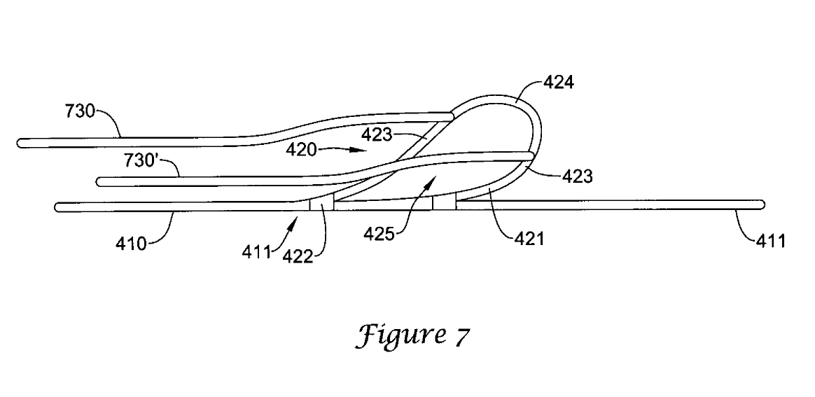

Turning to FIG. 7, another embodiment similar to the support structure of FIG. 6 is shown. In this embodiment, the support members can comprise elongate members (730, 730′). The elongate members (730, 730′) can be coupled to the first support hoop 420 in any of the configurations mentioned herein with respect to FIG. 6. Further, the elongate members (730, 730′) can extend proximally from the first support hoop 420 at an angle toward the first elongate member 410. At a point where the elongate members (730, 730′) are adjacent to the first elongate member 410, the second and third elongate members (730, 730′) can bend so that they extend proximally from the bend substantially parallel and adjacent the first elongate member 410. This configuration for the second and third elongate members (730, 730′) can further facilitate access to the mouth of the emboli capturing device. As described with respect to FIGS. 2 and 4, the frame of FIG. 6 can have a capturing basket disposed thereon.

In the embodiments of FIGS. 4-7, an alignment member (e.g., as described with respect to FIGS. 1A-2) can be incorporated into any of the support structures. Some of the embodiments can have an alignment member disposed in a proximal portion of the first elongate member (or any of the additional elongate members). For example, in the embodiments of FIGS. 4 and 5, the second elongate member 430 can be coupled to the top 424 of the first support hoop 420. The second elongate member 430 can extend proximally from this point, and can gradually reach a point at which it is adjacent the first elongate member 410. At this point, or proximal of this point, an alignment member can be used to keep these elongate members adjacent and/or parallel to one another.

A similar configuration can be used in FIG. 6. An alignment member that can accommodate three elongate members can be disposed in a proximal portion of the elongate members. For example, the alignment member can be disposed at a proximal point where the second and third elongate members are adjacent the first elongate member. Further, the alignment member can be coupled to and/or longitudinally fixed along and/or formed around the second and third elongate members and allow the first elongate member to translate longitudinally through the alignment member. The opposite is also contemplated; the alignment member can be longitudinally translatable along the second and third elongate members and it can be coupled to and/or longitudinally fixed along and/or or formed around the first elongate member. A similar alignment member can be used in FIG. 7. However, in some embodiments of FIG. 7, the alignment member can be disposed at a location immediately proximal the first elongate member distal end 411.

The embodiments of FIGS. 4-7 can also be used similarly to the methods of use described with respect to FIGS. 1A-1C and 2 above. Specifically, longitudinal movement of the second (or more) elongate members with respect to the first elongate member can move the first support hoop 420 and the capturing basket between collapsed and expanded configurations. As mentioned above, the first support hoop 420 of FIGS. 4-7 can be similar in any respect to the first support hoops of other embodiments described herein. In some cases, the first support hoop 420 can be predisposed to assume either a collapsed configuration or an expanded configuration.

The second (or more) support members of FIGS. 4-7 can be moved proximally, exert a pulling force on the first support hoop 420, moving the first support hoop 420 from a collapsed configuration to an expanded configuration. This pulling force can be used to overcome the predisposition of the first support hoop 420 to assume a collapsed configuration, and/or the pulling force can be used to maintain the first support hoop 420 in an expanded configuration. Also, the second (or more) support members of FIGS. 4-7 can be moved distally, exert a pushing force on the first support hoop 420, moving the first support hoop 420 from a collapsed configuration to an expanded configuration. In some cases, a pushing force can be used to overcome a predisposition of the first support hoop 420 to assume an expanded configuration.

Further, the emboli capturing embodiments shown in FIGS. 1-7 show a proximally facing emboli capturing device. It is also contemplated that the emboli capturing device can face distally, and the capturing membrane can extend proximally from the mouth of the filter. In such a case, the configuration and operation of the emboli capturing device can be reversed with respect to any or all aspects of the above embodiments.

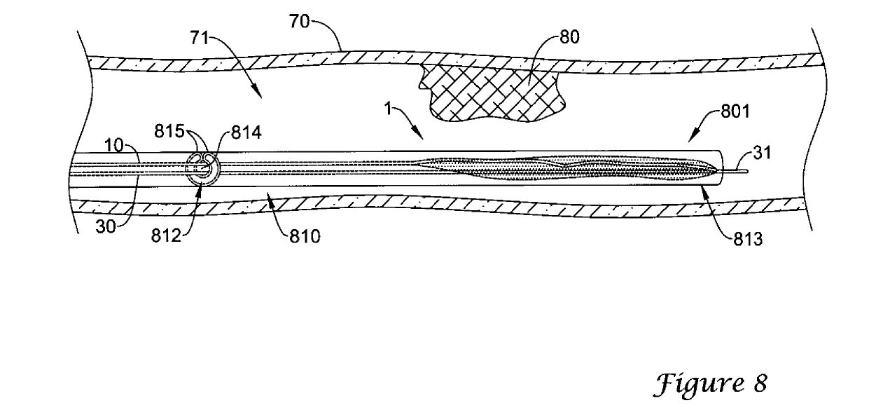

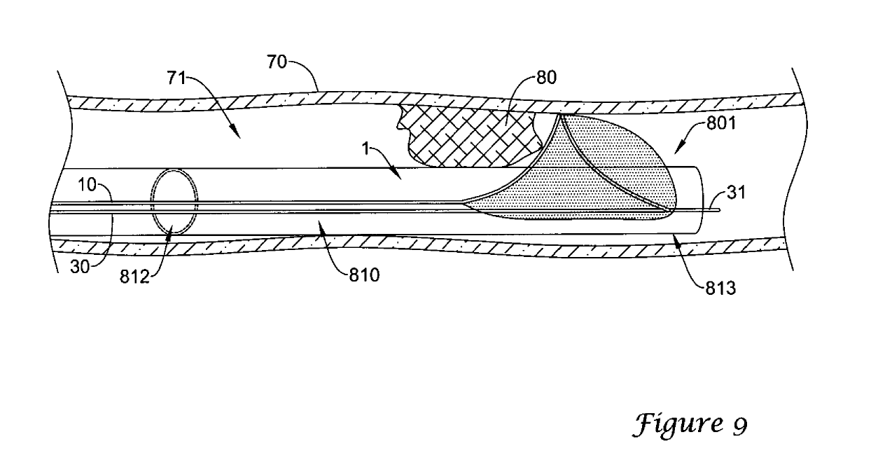

In some cases, emboli can form a partial or total occlusion of a vessel. In such cases, it can be desired to provide a flow of fluids (e.g., blood, therapeutic fluids, or both) around the occlusion. In FIGS. 8 and 9, one example of a combination device 801 comprising an emboli capturing device 1 and a perfusion lumen 810 is shown. Note that the perfusion lumen extends to the left of the drawings of FIGS. 8 and 9, and, for illustrative purposes, a cross-section of the perfusion lumen is shown in phantom in these figures.

Turning to FIG. 8, an example combination perfusion and emboli capture device is shown. This combination device 801 can have a perfusion member 810 with a distal end 813. The perfusion member 810 can also define a perfusion lumen 812. The perfusion member 810 can be disposed along the elongate members (10, 31) as further discussed below.

The perfusion member 810 can have a collapsed and an expanded configuration. In FIG. 8, the perfusion member 810 is shown in a collapsed configuration, which can form a relatively low profile compared to an expanded configuration (for example, the expanded configuration of FIG. 9, discussed below). In this embodiment, the perfusion member 810 is collapsed around the elongate members (10, 31) along at least a portion of the length of the elongate members (10, 31) from a proximal portion to a distal portion of the first elongate member 10 or from a proximal portion to a distal portion of the second elongate member 31, or both. Further, the perfusion member 810, when in a collapsed configuration, can be disposed around the first elongate member 10, the second elongate member 31, or both along the entire length of the first elongate member 10, the second elongate member 31, or both.

In some embodiments, the perfusion member 810 can be attached to the first elongate member 10, the second elongate member 31, or both, at one or more discrete points or along a portion or portions of the length of the first elongate member 10, the second elongate member 31, or both. In some cases, the perfusion member 810 can be attached to the first elongate member 10, the second elongate member 31, or both, along substantially the entire length of the perfusion member 810. In other embodiments, the perfusion member 810 can be longitudinally translatable with respect to the first elongate member 10, the second elongate member 31, or both. For example, at least a portion of the perfusion member 810 could be disposed around one of the elongate members (10, 31), but an inner surface 814 of the collapsed perfusion member 810 and an outer surface of one or both of the elongate members (10, 31) can be unattached.

In some cases where the perfusion member 810 and the elongate members (10, 31) are longitudinally translatable with respect to one another, the perfusion member 810, when in its collapsed configuration, can be advanced over the elongate members (10, 31). In such a case, the elongate members (10, 31) could be advanced through a patient's vasculature and the perfusion member 810 could then be advanced over the elongate members (10, 31). In addition, the perfusion member 810 could be advanced through a patient's vasculature first and then the elongate members (10, 31) passed down along (e.g., through) the collapsed perfusion member 810. Further, when the perfusion member 810 and the elongate members (10, 31) are longitudinally translatable with respect to one another, adjustments can be made in the positioning of the perfusion member distal end 813 with respect to the emboli capturing device 1, either before or after deploying the perfusion member 810 and/or the emboli capturing device 1.

In an alternative embodiment, the perfusion member 810, when in its collapsed configuration, can be disposed around the first elongate member 10, the second elongate member 31, or both, along only a portion of the length of the respective elongate member(s). For example, when the perfusion member 810 is in its collapsed configuration, a distal portion of the perfusion member 810 can be disposed around the first elongate member 10, the second elongate member 31, or both. Further, a proximal portion of the perfusion member 810 can be disposed alongside the first elongate member 10, the second elongate member 31, or both. In some cases, the perfusion member 810 can be longitudinally translatable with respect to the first elongate member 10, the second elongate member 31, or both. In some embodiments, if a distal portion of the perfusion member 810 is disposed around one or both of the elongate members (10, 31) and a proximal portion of the perfusion member 810 is disposed along side the elongate members (10, 31), the perfusion member 810 can be longitudinally translatable with respect to the elongate members (10, 31) in a single operator exchange type fashion.

Further, the perfusion member 810 can comprise a variety of materials. In one embodiment, the perfusion member 810 can made from a material that is elastic in at least a radial direction about an axis running the length of the perfusion member 810. With the perfusion member 810 being an elastic member, it can form different size lumens and/or accommodate different flow rates by expanding to allow for higher flow rates. In other embodiments, the perfusion member 810 can be inelastic in a radial direction about an axis running the length of the perfusion member 810, which can ensure that it will not expand beyond a certain size when it deploys. In such a case, the perfusion member 810 may be sized for a certain application. One of ordinary skill in the art would recognize the instances when either or both of these types of designs would be suitable.

Turning again to FIG. 8, the perfusion member 810 can be formed into the collapsed configuration in any number of ways. For example, an inner surface 814 of the collapsed perfusion member 810 could be adhered to either one or both of the elongate members (10,31) along all or a portion of the inner surface 814. Such adhesion could be by using adhesive, by heating and partially melting one or both of the members, or by other means known to those of skill in the art. The adhesion can be of sufficient strength to maintain the perfusion member 810 around one or both of the elongate members (10, 31) as the device 1 is being manipulated within a patient's vasculature. Also, the strength of the adhesion can be sufficiently weak to allow the perfusion member 810 to partially or totally release from either one or both of the elongate members (10, 31) when the perfusion member 810 is translated from a collapsed to an expanded configuration. This can allow for the perfusion member 810 to be securely in place around either one or both of the elongate members (10, 31) in a collapsed configuration and, when expanded, it can be released to define an open perfusion lumen 812, as shown in FIG. 9.

In other embodiments, the perfusion member 810 can be predisposed to assuming the collapsed position. In such a case, the perfusion member 810 can be disposed around either one or both of the elongate members (10, 31), and the predisposition of the perfusion member 810 can cause the perfusion member 810 to wrap around one or both of the elongate members (10, 31). For example, the perfusion member 810 can form into a folded “C” shape shown in FIG. 8. In other cases, all or a portion of the length of the perfusion member 810 can have a bi-stable or limited-stability element disposed in it. For example, this bi-stable or limited-stability element can be predisposed to assuming a is first shape, a second shape, or both. For example, a bi-stable or limited-stability element can be predisposed to assuming a collapsed configuration, and in the process this element can cause the perfusion member 810 to also assume a collapsed configuration (e.g., if the element were disposed within the perfusion member 810, if the element were adhered to the perfusion member 810, or both). Similarly, the bi-stable or limited-stability element can be predisposed to assuming an expanded configuration, and in the process this element can cause the perfusion member 810 to also assume an expanded configuration. In other embodiments, the bi-stable or limited-stability element can be predisposed to assume the first shape and, if deformed past a certain point, it can be predisposed to assume the second shape.

In addition, other embodiments are also envisioned in which the perfusion member 810 can be disposed about one or both of the elongate members (10, 31). For example, turning again to FIG. 8, the folded portions 825 of the perfusion member 810 can be in close proximity to one another, forming the perfusion member 810 into a “C” shape around either one or both of the elongate members (10, 31). In some cases, the folded portions 825 can be attached in order to dispose the perfusion member 810 around either one or both of the elongate members (10, 31). If these folded portions 825 are attached, the perfusion member 810 can in some cases essentially form a lumen in which either one or both of the elongate members (10, 31) can be disposed. The attachment of these folded portions can be by stitching them to one another, by bringing them in close proximity and using an adhesive and/or heat and/or laser welding them to one another, or by other suitable means.

In some cases, when using a bi-stable or limited-stability element, when attaching the folded portions 825 to one another, or when using other suitable methods of disposing the perfusion member 810 about the elongate members (10, 31), the perfusion member 810 and the elongate members (10, 31) can remain unattached to one another. In some cases, the perfusion member 810 and the elongate members (10, 31) can be longitudinally translatable with respect to one another.

Turning to FIG. 9, a perspective view of a combination device 801 is shown with the perfusion member 810 and the emboli capturing device 1 both in expanded configurations. In this figure, the perfusion member 810 and the emboli capturing device 1 are eccentrically disposed along the second elongate member 31. In other examples, the perfusion member 810 and the emboli capturing device 1 can be eccentrically disposed along the first elongate member 10. Further, one of the perfusion member 810 and emboli capturing device 1 can be eccentrically disposed along the first elongate member 10 and the other can be eccentrically disposed along the second elongate member 31. In other embodiments, the perfusion member 810 can be disposed along one of the first or second elongate members (10, 31) and the emboli capturing device 1 can be disposed eccentrically along both the first and second elongate members (10, 31).

In some cases, when the perfusion member 810 and the emboli capturing device 1 are both in expanded configurations, the cross-sections of the perfusion member 810 and the emboli capturing device 1 can combine to partially or substantially completely fill a cross-section of the open area of a patient's body vessel in at least one longitudinal portion of the body vessel. In such a cases, flow of bodily fluids through the body vessel can be partially or entirely prevented other than any flow through the emboli capturing device and/or through the perfusion member. In some embodiments, the perfusion member 810 and the emboli capturing device 1 can be configured to fit together in such a way that the cross-section of the combination device 801 in its expanded configuration can be circular, or any other shape that corresponds to a body vessel in which it will be placed.

As mentioned above, the emboli capturing device 1 can partially or entirely occlude a body vessel. In some examples, the emboli capturing device 1 can, when in an expanded configuration, occlude 30% or more, 40% or more, 50% or more, or 75% or more of the cross-section of a body vessel. The remainder of the cross-section of the body vessel lumen can be occupied by the expanded or collapsed perfusion member and the elongate support members. Alternatively, and as mentioned above, some embodiments can allow for the emboli capturing device 1 to be advanced independently of the perfusion member 810. In such a case, the emboli capturing device 1 can be deployed to an expanded configuration and can substantially occlude the entire cross-section of a body vessel. Also, in some embodiments, the emboli capturing device 1 can be deployed into an expanded configuration while the perfusion member 810 remains substantially or entirely in a collapsed configuration. In such a case, the emboli capturing device 1 can occlude a larger fraction of the cross-section of a body vessel, for example 50% or more, 60% or more, 70% or more, 75% or more, 80% or more, 90% or more, or 95% or more of the cross-section of the body vessel.

Other configurations are also possible for the emboli capturing device and the perfusion member relative to one another within the combination device 801. Some examples of alternate configurations can be found in patent application Ser. No. 11/693,956 (Attorney Docket No. 1001.1971101), entitled “PERFUSION AND EMBOLIC PROTECTION,” which was filed Mar. 30, 2007. This reference is herein incorporated in its entirety.

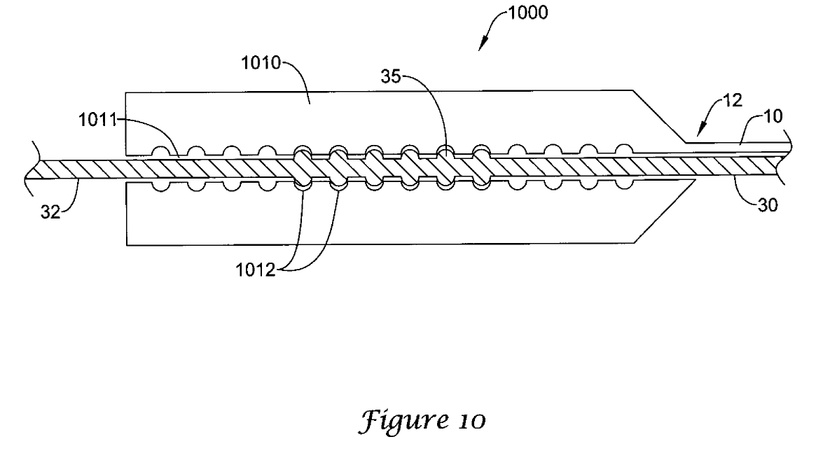

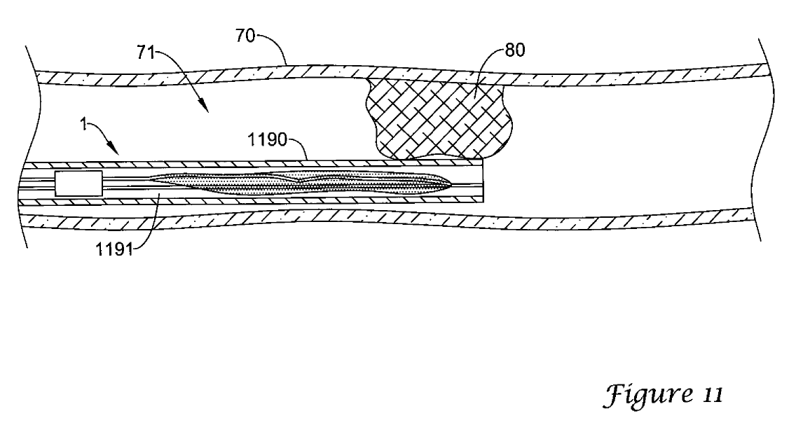

In some cases, it may be desired, when moving the elongate members longitudinally with respect to one another, to position the members at predetermined longitudinal positions with respect to one another. In some cases, it may also be desirable to fix (i.e., temporarily fix) the members at certain longitudinal positions with respect to one another. Turning now to FIG. 10, a cross-section of an example of a hub for an emboli capturing device is shown. The hub can be designed to provide for one or more indexing positions or stops at predetermined longitudinal positions. The hub 1000 can comprise a hub body 1010. The hub body 1010 can define a lumen 1011 in which one of the first or second elongate members (10, 31) can be disposed. The elongate member (10, 31) that is not disposed in the lumen 1011 can be coupled to the hub body 1010. In some embodiments, the first elongate member proximal end 12 can be coupled to the hub body 1010 (e.g., it can be coupled using adhesive, welding, soldering, or any other suitable methods of attachment), and in other cases the first elongate member proximal end 12 can be integral with the hub body 1010 (egg., the hub body 1010 can be formed around the proximal end 12 or the hub body 1010 and the proximal end 12 can be formed together).

The hub body 1010 and the elongate member that is disposed in the hub body lumen 1011 can coordinate or interlock with one another. In some cases, the elongate i5 member and the hub body 1011 can coordinate or interlock at multiple different longitudinal positions, providing an indexing and/or locking mechanism for positioning and/or maintaining the first and second elongate members (10, 31) at certain positions with respect to one another. The coordination between the elongate member and the hub body 1010 can take a variety of forms, including coordinating shapes on the elongate member and the wall of the hub body lumen 1011, or other mechanisms such as a luer compression fitting disposed on the hub body, or other suitable mechanisms for interlocking the elongate member and the hub body 1010.

In FIG. 10, the second elongate member 31 is disposed within the hub body lumen 1011. The hub body lumen comprises a series of indentations 1012 formed in the wall of the hub body lumen 1011. Further, the second elongate member 31 is shown having protrusions 35 that can be disposed along the second elongate member 31 such that they can coordinate with the indentations 1012 of the wall of the hub body lumen 1011. In other cases, the second elongate member 31 can have indentations and the wall of the hub body lumen 1011 can have protrusions that can interact, or both the second elongate member 31 and the wall of the hub body lumen 1011 can have protrusions that can interact.

The hub body 1010 can be shaped and sized in order to allow an operator to grasp the hub body 1010. Further, the second elongate member proximal end 33 can simply comprise the end of the second elongate member or the second elongate member proximal end 33 can have a hub or other structure formed on it in order to allow an operator to more easily grasp and manipulate the elongate member that extends through the hub lumen 1011.

FIG. 10 shows the second elongate member 31 being disposed within the hub body lumen 1011. However, as mentioned above, the first elongate member 10 can be disposed in the hub body lumen 1011 and the second elongate member 31 can be coupled to the hub body 1010, in which case the first elongate member 10 can be shaped and configured to interact with the hub body lumen 1011 as described above with respect to the second elongate member 31.

Other methods of positioning one elongate member with respect to another are also known in the art, and any of these mechanisms could be incorporated into hub body in place of, or in addition to, the mechanisms shown in FIG. 10.

In addition, markers could be used on the exposed, proximal end of the elongate member that is disposed within the hub body lumen 1011. The markers could indicate positions at which the emboli capturing device is in an expanded configuration, a collapsed configuration, a partially expanded configuration, or any combination thereof For example, the markers could be numbers, letters, words, shapes, symbols or colors. Such markers could be combined with any of the hub systems discussed herein.

Further, a hub system can also be adapted to accommodate an emboli capturing device that comprises more than two elongate members. In cases where a first elongate member is coupled to the base of a first support hoop and second and third (or more) elongate members are coupled to the first support hoop at a location other than the base, the hub body can accommodate the first elongate member in a hub body lumen and the second and third (or more) elongate members can be coupled to the hub body. In the alternative, the hub body can accommodate the second and third (or more) elongate members in a single hub body lumen or in multiple hub body lumens (e.g., each elongate member could have its own hub body lumen) and the first elongate member can be coupled to the hub body. In addition, if the second and third (or more) elongate members are disposed in one or more hub body lumens, these elongate members can be attached to one another in order to ensure that they are moved longitudinally in tandem. The attachment point can be proximate the first support hoop, proximate the hub, or at any position therebetween, or within or proximal of the hub.

FIG. 11 shows an emboli capturing device 1 that is in a collapsed configuration inside a sheath 1190. The sheath 1190 can define a sheath lumen 1191. The sheath lumen 1191 can have an inside diameter that is sized and configured to contain the emboli capturing device 1, for example when the emboli capturing device 1 is in a collapsed configuration. The sheath 1190 can be used in conjunction with any of the emboli capturing devices or in conjunction with any of the combination emboli capturing/perfusion devices described herein.

In one example shown in FIG. 11, the emboli capturing device 1 along with the sheath 1190 could be advanced through a vessel lumen 71 to adjacent a treatment site, for example adjacent an embolus 80. In some cases, an emboli capturing device 1 in a collapsed configuration and within a sheath 1190 can be advanced to a position proximal, distal, and/or adjacent the treatment site. The sheath 1190 could then be pulled proximally and/or the embolic capturing device 1 could be pushed distally. In addition, the emboli capturing device 1 could then be placed in an expanded configuration and used to perform a procedure, for example as described above with respect to FIGS. 3B and 3C.

The elongate members and the support hoops described herein may be formed of any suitable materials, dependent upon the desired properties of these structures. Some examples of suitable materials include metals, metal alloys, polymers, composites, or the like, or combinations or mixtures thereof. Some examples of suitable metals and metal alloys include stainless steel, such as 304V, 304L, and 326L stainless steel; alloys including nickel-titanium alloy such as linear elastic or superelastic (i.e., pseudoelastic) nitinol; nickel-chromium alloy; nickel-chromium-iron alloy; cobalt alloy; tungsten or tungsten alloys; MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); hastelloy; monel 400; inconel 625; or the like; or other suitable material, or combinations or alloys thereof. In some embodiments, it is desirable to use metals or metal alloys that are suitable for metal joining techniques such as welding, soldering, brazing, crimping, friction fitting, adhesive bonding, etc. The particular material used can also be chosen in part based on the desired flexibility requirements or other desired characteristics.

Some examples of suitable polymers can include, but are not limited to, polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro(propyl vinyl ether) (PFA), polyether-ester, some adhesive resin such as modified polyolefin resin, polymer/metal composites, etc., or mixtures, blends or combinations thereof, and may also include or be made up of a lubricous polymer. Some other potentially suitable polymer materials may include those listed herein with reference to other components of the catheter 10. One example of a suitable polyether block ester is available under the trade name ARNITEL, and one suitable example of a polyether block amide (PEBA) is available under the trade name PEBAX®, from Atomchem Polymers, Birdsboro, Pa. In some embodiments, adhesive resins may be used, for example, as tie layers and/or as the material of the structures. One example of a suitable adhesive resin is a modified polyolefin resin available under the trade name ADMER®, from Mitsui Chemicals America, Inc. Additionally, polymer material can, in some instances, be blended with a liquid crystal polymer (LCP). For example, in some embodiments, the mixture can contain up to about 5% LCP. This has been found in some embodiments to enhance torqueability. Components of the catheter 20, such as the elongate support member 30 201, the additional tubular member and/or coating 203, the inflation tube 210, the guidewire tube 220, or any combination thereof can incorporate any of the above polymers.

In some embodiments, the elongate members can comprise stainless steel. In other embodiments, a proximal portion of the elongate members can comprise one material (e.g., stainless steel) and a distal portion can comprise another material (e.g., superelastic or linear elastic Nitinol). The support hoop(s) can comprise, for example, stainless steel or Nitinol (either superelastic or linear elastic). Further, in cases where an elongate member is integral with a support hoop of support strut, the elongate member can comprise a wire that comprises all Nitinol (either superelastic or linear elastic), or stainless steel in a proximal portion of the wire and Nitinol (either superelastic or linear elastic) in a distal portion (e.g., the distal end) of the wire. The distal portion of the wire can be used for forming a support strut or support hoop, thus forming the strut or hoop from Nitinol.

The elongate members can have a variable flexibility along its length, for example they can be more flexible in a distal region compared to a proximal region. In some embodiments, such a change in flexibility can result from a variation in the cross-sectional area of the elongate members along its length or a variation in the material of construction, or both. For example, the elongate members can each comprise a single elongate member that varies in composition or other properties such as cross-sectional area or shape along its length (e.g., as described above) and/or it can comprise multiple elongate members that are joined to one another and that differ in composition or in other properties such as cross-sectional area or shape. Further, the elongate members can have a round cross-sectional shape. The elongate members could also have other cross-sectional shapes; for example, the cross-sectional shape could be square, triangular, rectangular, oval, polygonal, or other shapes, or the cross-sectional shape could vary along the length of the elongate members.

Additionally, in some instances a degree of MRI compatibility can be imparted into the devices of this application. For example, to enhance compatibility with Magnetic Resonance Imaging (MRI) machines, all or portions of the elongate members, the emboli capturing devices or the combination emboli capturing and perfusion devices can be made in a manner that would impart a degree of MRI compatibility. For example, all or a portion of the structures may be made of a material that does not substantially distort the image and create substantial artifacts (artifacts are gaps in the image) during MRI imaging. Certain ferromagnetic materials, for example, may not be suitable because they may create artifacts in an MRI image. All or a portion of the structures may also be made from a material that the MRI machine can image. Some materials that exhibit these characteristics include, for example, tungsten, Elgiloy, MP35N, nitinol, and the like, and others, or combinations or alloys thereof.

Further, any of the embodiments described herein can be used to cross lesions (e.g., emboli) in a body vessel. In order to cross lesions, it may be required to puncture through the lesion or otherwise expand any open area of the vessel in order to allow the devices of this application to pass through distally of the lesion. In such cases, the distal-most end of any of the devices in this application can be formed of a relatively stiff construction in order to allow the lesion to be crossed, for example by forcing the relatively stiff portion through the lesion or the small opening on the vessel.

We claim:

1. An emboli capturing device comprising:
a first elongate member with a distal portion;
a first support hoop coupled to the first elongate member distal portion, the first support hoop having a collapsed configuration and an expanded configuration;
a capture membrane disposed on the support hoop forming a capture basket; and
a first support member comprising a second elongate member having a distal end, the first and second elongate members being longitudinally translatable with respect to one another;
wherein the first elongate member is coupled to the first support hoop at a first location on the first support hoop and the first support member is coupled to the first support hoop at a second location on the first support hoop.

2. The device of claim 1, wherein the second elongate member has first distal and second proximal longitudinal positions with respect to the first elongate member, wherein moving the second elongate member between the first and second longitudinal positions translates the first support hoop between collapsed and expanded configurations.

3. The device of claim 2, wherein moving the second elongate member from the first to the second longitudinal position translates the first support hoop from the collapsed to the expanded configuration.

4. The device of claim 2, wherein moving the second elongate member from the first to the second longitudinal position translates the first support hoop from the expanded to the collapsed configuration.

5. The device of claim 1, wherein the second elongate member is coupled to the first support hoop.

6. The device of claim 1, wherein the first support member further comprises a support strut with a first and a second end, the strut being coupled to the first support hoop at the first end and to the second elongate member at the second end.

7. The device of claim 1, wherein the first support member further comprises a second support hoop, and the second support hoop has a base that is coupled to the second elongate member and another portion of the second support hoop is coupled to the first support hoop.

8. The device of claim 1, comprising a second support member comprising a third elongate member, the second and the third elongate members being coupled to the first support hoop.

9. The device of claim 8, wherein the first elongate member is coupled to a base of the first support hoop, the first support member is coupled to the first support hoop at a first side of the first support hoop and the second support member is coupled to the first support hoop at a second side of the first support hoop.

10. The device of claim 8, wherein a distal portion of the second and third elongate members extend proximally from the first support hoop at an angle toward the first elongate member to a proximal point where they are adjacent the first elongate member, and extend from this proximal point proximally along the first elongate member.

11. The device of claim 1, further comprising an alignment member defining a lumen, the alignment member longitudinally fixed along one of the elongate members and having another of the elongate members extending through the lumen.

12. The device of claim 1, further comprising an alignment member defining a lumen through which both of the first and second elongate members extend.

13. The device of claim 1, further comprising an alignment member comprising a tube defining a lumen, wherein both of the elongate members extend through the lumen.

14. The device of claim 1, further comprising an alignment member defining a first and second lumen, wherein the first elongate member extends through the first lumen and the second elongate member extends through the second lumen.

15. The device of claim 1, further comprising an alignment member, the alignment member longitudinally fixed along one of the elongate members.

16. The device of claim 1, wherein the capture membrane is non-porous.

17. The device of claim 1, wherein the capture membrane is a filter membrane having pores.

18. The device of claim 1, further comprising a perfusion member having collapsed and expanded configurations, the perfusion member being wrapped around the first and second elongate members when in the collapsed configuration and defining a perfusion lumen when in the expanded configuration.

19. The device of claim 1, wherein the first elongate member has a distal end and the first support hoop has a base coupled to the distal end.

20. An emboli capturing device comprising:
a first elongate member with a distal portion;
a first support hoop having a base, two sides and a top, the base being coupled to the first elongate member distal portion, the first support hoop having a collapsed configuration and an expanded configuration;
a capture membrane disposed on the support hoop forming a capture basket; and
a first support member comprising a second elongate member having a distal end, the first and second elongate members being longitudinally translatable with respect to one another;
wherein, when the first support hoop is in the collapsed configuration, the first support member is coupled to the first support hoop at a point distal of the first support hoop base.

21. The device of claim 20, wherein the first support member is coupled to at least one of the sides of the first support hoop.

22. The device of claim 20, wherein the first support member is coupled to the top of the first support hoop.

23. The device of claim 20, wherein the second elongate member has first distal and second proximal longitudinal positions with respect to the first elongate member, wherein moving the second elongate member between the first and second longitudinal positions translates the first support hoop between collapsed and expanded configurations.

24. The device of claim 23, wherein moving the second elongate member from the first to the second longitudinal position translates the first support hoop from the collapsed to the expanded configuration.

25. The device of claim 23, wherein moving the second elongate member from the first to the second longitudinal position translates the first support hoop from the expanded to the collapsed configuration.

26. The device of claim 20, wherein the first support member further comprises a support strut with a first and a second end, the strut being coupled to the first support hoop at the first end and to the second elongate member at the second end.

27. The device of claim 20, wherein the first support member further comprises a second support hoop, and the second support hoop has a base that is coupled to the second elongate member and another portion of the second support hoop is coupled to the first support hoop.

28. The device of claim 20, comprising a second support member comprising a third elongate member, the second and third elongate members being coupled to the first support hoop.

29. The device of claim 28, wherein the first elongate member is coupled to a base of the first support hoop, the first support member is coupled to the first support hoop at a first side of the first support hoop and the second support member is coupled to the first support hoop at a second side of the first support hoop.

30. The device of claim 28, wherein a distal portion of the second and third elongate members extend proximally from the first support hoop at an angle toward the first elongate member to a proximal point where they are adjacent the first elongate member, and extend from this proximal point proximally along the first elongate member.

31. The device of claim 20, further comprising an alignment member defining a lumen, the alignment member longitudinally fixed along one of the elongate members and having another of elongate members extending through the lumen.

32. The device of claim 20, further comprising an alignment member defining a lumen through which both of the first and second elongate members extend.

33. The device of claim 20, further comprising an alignment member comprising a tube defining a lumen, wherein both of the elongate members extend through the lumen.

34. The device of claim 20, further comprising an alignment member defining a first and second lumen, wherein the first elongate member extends through the first lumen and the second elongate member extends through the second lumen.

35. The device of claim 20, further comprising an alignment member, the alignment member longitudinally fixed along one of the elongate members.

36. The device of claim 20, wherein the capture membrane is non-porous.

37. The device of claim 20, wherein the capture membrane is a filter membrane having pores.

38. The device of claim 20, further comprising a perfusion member having collapsed and expanded configurations, the perfusion member being wrapped around the first and second elongate members when in the collapsed configuration and defining a perfusion lumen when in the expanded configuration.

39. A method of capturing embolic material comprising:
providing an embolic capturing device comprising a first elongate member with a distal portion, a first support hoop attached to the first elongate member distal portion, the first support hoop having a collapsed configuration and an expanded configuration, a capture membrane disposed on the support hoop forming a capture basket, a support member comprising a second elongate member having a distal end, the first and second elongate members being longitudinally translatable with respect to one another, wherein the first elongate member is coupled to the first support hoop at a first location and the support member is coupled to the first support hoop at a second location;
introducing the embolic capturing device into a patient's vasculature;
advancing the embolic capturing device so that the capture basket is distal of an emboli in a patient's vasculature;
translating the embolic capturing device from a collapsed configuration to an expanded configuration;
moving an open end of the embolic capturing device toward the emboli; and
capturing the emboli in the capturing device.

40. The device of claim 39, wherein the emboli is adhered to an inner surface of the patient's vasculature, and wherein the step of capturing the emboli in the capturing device includes using the emboli capturing device to separate the emboli from the inner surface.

41. The device of claim 39, further comprising the step of providing a perfusion member that has collapsed and expanded configurations, the perfusion member being wrapped around the first and second elongate members when in the collapsed configuration and defining a perfusion lumen when in the expanded configuration, wherein the method further includes the steps of;
introducing the perfusion member into a patient's vasculature in the collapsed configuration; and
translating the perfusion member from the collapsed configuration to the expanded configuration.

42. The device of claim 41, wherein the step of translating the perfusion member from the collapsed configuration to the expanded configuration is performed after the step of translating the capturing device from an expanded configuration to a collapsed configuration.

43. The device of claim 41, wherein the step of translating the perfusion member from the collapsed configuration to the expanded configuration is performed before the step of translating the capturing device from an expanded configuration to a collapsed configuration.

44. The device of claim 41, wherein the perfusion member is longitudinally translatable along the elongate members and the method further includes the step of advancing the perfusion member along the emboli capturing device after the emboli capturing device is within the patient's vasculature.

45. The device of claim 41, wherein the perfusion member is longitudinally translatable along the elongate members and the method further includes the step of introducing the perfusion member into the patient's vasculature and thereafter introducing the emboli capturing device into the patient's vasculature along the perfusion member.

46. The device of claim 39, further comprising the steps of translating the capturing device from an expanded configuration to a collapsed configuration and removing the emboli capturing device from the patient along with the captured emboli.

47. A combination perfusion and emboli capturing device comprising:
an emboli capturing device comprising:
a first elongate member with a distal portion;
a first support hoop coupled to the first elongate member distal portion, the first support hoop having a collapsed configuration and an expanded configuration;
a capture membrane disposed on the support hoop forming a capture basket; and
a first support member comprising a second elongate member having a distal end, the first and second elongate members being longitudinally translatable with respect to one another;
wherein the first elongate member is coupled to the first support hoop at a first location and the first support member is coupled to the first support hoop at a second location; and
a perfusion member having collapsed and expanded configurations, the perfusion member being wrapped around the first and second elongate members when in the collapsed configuration and defining a perfusion lumen when in the expanded configuration.

48. The device of claim 475 wherein the perfusion member is longitudinally translatable along the first and second elongate members when the perfusion member is in its collapsed configuration.

49. The device of claim 47, wherein, when both the perfusion member and the emboli capturing device are in an expanded configuration, both the perfusion member and the emboli capturing device are eccentrically disposed along the first and second elongate members.
